(19) United States
(12) Patent Application Publication
(10) Pub. No.: US 2017/0002370 A1
(43) Pub. Date: Jan. 5, 2017

(54) ISOLATION OF TERMINATOR SEQUENCES CAUSING ENHANCED TRANSCRIPTION

(71) Applicant: BASF PLANT SCIENCE GMBH, Ludwigshfen (DE)

(72) Inventor: Huihua Fu, Cary, NC (US)

(21) Appl. No.: 15/104,705

(22) PCT Filed: Dec. 10, 2014
(86) PCT No.: PCT/IB2014/066749

§ 371 Date: Jun. 15, 2016

Related U.S. Application Data
(60) Provisional application No. 61/918,828, filed on Dec. 20, 2013.

(51) Int. Cl. C12N 15/82

(52) U.S. Cl. CPC C12N 15/8216; C12N 15/8257

(57) ABSTRACT

Disclosed herein are heterologous DNA constructs comprising one or more terminator sequences causing enhanced transcription.

This application claims priority to 61/918,828 filed Dec. 20, 2013, which is incorporated herein by reference in their entirety.

### FIELD OF THE INVENTION

The invention relates to heterologous DNA constructs comprising a terminator sequence causing enhanced transcription.

### BACKGROUND OF THE INVENTION

The aim of plant biotechnology is the generation of plants with advantageous novel properties, such as pest and disease resistance, resistance to environmental stress (e.g., water-logging, drought, heat, cold, light-intensity, day-length, chemicals, etc.), improved qualities (e.g., high yield of fruit, extended shelf-life, uniform fruit shape and color, higher sugar content, higher vitamins C and A content, lower acidity, etc.), or for the production of certain chemicals or pharmaceuticals (Dunwell, 2000). Furthermore resistance against abiotic stress (drought, salt) and/or biotic stress (insects, fungi, nematode infections) can be increased. Crop yield enhancement and yield stability can be achieved by developing genetically engineered plants with desired phenotypes.

For all fields of biotechnology, beside promoter sequences, terminator sequences positioned in the 3′UTR of genes are a basic prerequisite for the recombinant expression of specific genes. In animal systems, a machinery of transcription termination has been well defined (Zhao et al., 1999; Proudfoot, 1986; Kim et al., 2003; Yonaha and Proudfoot, 2000; Cramer et al., 2001; Kuerstem and Goodwin, 2003).

However, terminators may have more effects than simple termination of transcription. For example, by Narsai et al. it has been reported that terminators house mRNA stabilizing or de-stabilizing elements (Narsai et al. 2007). Among these may be AU-rich elements or miRNA binding sites. Furthermore, efficient termination may serve to free the transcribing polymerase to recycle back to the transcriptional start site. This may cause higher levels of transcriptional initiation (Nagaya et al. 2010.; Mapendano et al. 2010). 3′ end processing of an mRNA can also be involved in nuclear export and subsequent translation. Therefore, terminator sequences can influence the expression level of a transgene positively or negatively.

Mutagenesis experiments of plant terminators have identified three major elements: far upstream elements (FUEs), near upstream elements (NUEs; AAUAAA-like motifs) and a cleavage/polyadenylation site (CS). The NUE region is an A-rich element located within 30 nt of the poly(A) site (Hunt, 1994). The FUE region is a U- or UG-richsequence that enhances processing efficiency at the CS (Mogen et al. 1990, Rothnie, 1996), which is itself a YA (CA or UA) dinucleotide within a U-rich region at which polyadenylation occurs (Bassett, 2007).

In plant biotech transgenes need often be expressed at high levels to cause a desired effect. For most applications the strength of the promoter is considered to primarily decide over the expression levels of a transgene. Promoter identification and characterization is costly and time-consuming as every promoter has its own specific expression pattern and strength. Furthermore, the set of strong suitable promoters is limited. With modern plant biotechnology proceeding towards stacking multiple expression cassettes in one construct, doubling of identical strong promoter elements for optimal gene expression bears the risk of homologous recombination or transcriptional gene silencing. However, using a weaker promoter as an alternative to circumvent this problem may limit the expression of one gene in the construct. This in turn may impair generating the desired phenotype in the transgenic plant. Instead of strong promoters, terminator sequences may be used to achieve enhanced expression levels of the gene they are functionally linked to. This allows use of alternative weaker promoters in a multigene-construct without compromising overall expression levels. A further advantage may be that promoter specificity and expression patterns are not changed if only the terminator is modified.

It is therefore an objective of the present invention, to provide a method to identify, isolate and test terminator sequences that enhance gene expression in plants. It is also an objective of the invention to provide such terminators that enhance gene expression. A further objective of the invention is to provide terminators efficiently terminating transcription.

### BRIEF DESCRIPTION OF THE FIGURES

FIG. 1: Nuceleic Acid Sequences for Terminators 1 and 2 and T-NOS

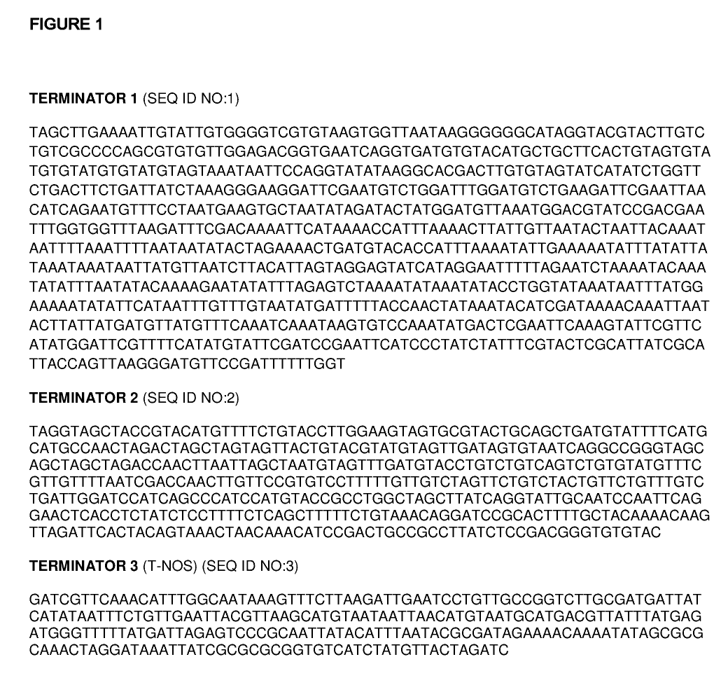

The nucleic acid sequences for Terminator 1 (SEQ ID NO:1), Terminator 2 (SEQ ID NO:2) and the prior art terminator t-nos (SEQ ID NO:3).

FIG. 2: MUG fluorescence in transgenic events

MUG fluorescence in T₀ seedling leaf tissue (Mean±SD). Each bar represents the average activity in multiple single-copy transgenic events (n=12, construct level). Activity is expressed in nanomoles of 4-methyl umbelliferone per minute per microgram protein.

### DETAILED DESCRIPTION OF THE INVENTION

An embodiment of the invention is an expression construct comprising a terminator molecule having a polynucleotide sequence as defined in SEQ ID NO:1 (Terminator 1) or SEQ ID NO:2 (Terminator 2), or the complement or reverse complement of SEQ ID NO:1, or the complement or reverse complement of SEQ ID NO:2, wherein the terminator molecule causes enhanced expression of heterologous nucleic acid molecules to be expressed to which the terminator molecule is functionally linked.

In a preferred embodiment, the terminator molecule is functionally linked to a nucleic acid molecule heterologous to the terminator molecule of the invention. Such nucleic acid molecule may for example be any regulatory nucleic acid molecule such as a promoter, a NEENA or an intron. The nucleic acid molecule may also be any nucleic acid to be expressed such as a gene of interest, a coding region or a noncoding region, for example a 5′ or 3′ UTR, a microRNA, RNAi, antisense RNA and the like.

Another embodiment of the invention is an expression construct comprising a terminator molecule comprising a sequence selected from the group consisting of
a) a nucleic acid molecule comprising the sequence of SEQ ID NO: 1 or 2, or
b) a nucleic acid molecule comprising a sequence having at least 60% sequence identity to SEQ ID NO: 1 or 2, 70% sequence identity to SEQ ID NO: 1 or 2, 80 sequence identity to SEQ ID NO: 1 or 2, 85% sequence identity to SEQ ID NO: 1 or 2, 90% sequence identity to SEQ ID NO: 1 or 2, 95% sequence identity to SEQ ID NO: 1 or 2, 96% sequence identity to SEQ ID NO: 1 or 2, 97% sequence identity to SEQ ID NO: 1 or 2, 98% sequence identity to SEQ ID NO: 1 or 2, 99% sequence identity to SEQ ID NO: 1 or 2, or
c) a fragment of 100 or more consecutive bases, 150 or more consecutive bases, 200 consecutive bases or more, or 250 or more consecutive bases of a nucleic acid molecule of a) or b) where the fragment has an expressing enhancing activity that is 65% or more, 70% or more, 75% or more, 80% or more, 85% or more or 90% or more, or 95% or more of the expression enhancing activity as a nucleic acid molecule comprising the sequence of SEQ ID NO: 1 or 2, or
d) a nucleic acid molecule which is the complement or reverse complement of any of the nucleic acid molecules under a) or b), or
e) a nucleic acid molecule of 100 nucleotides or more, 150 nucleotides or more, 200 nucleotides or more or 250 nucleotides or more, hybridizing under conditions equivalent to hybridization in 7% sodium dodecyl sulfate (SDS), 0.5 M NaPO4, 1 mM EDTA at 50° C. with washing in 2×SSC, 0.1% SDS at 50° C. or 65° C., to a nucleic acid molecule comprising at least 50, at least 100, at least 150, at least 200, at least 250 consecutive nucleotides of a transcription enhancing terminator of SEQ ID NO: 1 or 2 or the complement thereof. Said nucleic acid molecule hybridizes under conditions equivalent to hybridization in 7% sodium dodecyl sulfate (SDS), 0.5 M NaPO₄, 1 mM EDTA at 50° C. with washing in 1×SSC, 0.1% SDS at 50° C. or 65° C., to a nucleic acid molecule comprising at least 50, at least 100, at least 150, at least 200, at least 250 consecutive nucleotides of a transcription enhancing terminator of SEQ ID NO: 1 or 2 or the complement thereof, said nucleic acid molecule hybridizes under conditions equivalent to hybridization in 7% sodium dodecyl sulfate (SDS), 0.5 M NaPO4, 1 mM EDTA at 50° C. with washing in 0.1×SSC, 0.1% SDS at 50° C. or 65° C., to a nucleic acid molecule comprising at least 50, at least 100, at least 150, at least 200, at least 250 consecutive nucleotides of a transcription enhancing terminator of SEQ ID NO: 1 or 2,
   wherein the terminator molecules as defined under a) to e) cause enhanced expression of heterologous nucleic acid molecules to which they are functionally linked.

The nucleic acid molecules as defined above under b) to e) are functional enhancing terminator molecules, hence are terminating transcription and are having at least 50% of the expression enhancing effect of the respective molecule as defined above under a).

A further embodiment of the invention is a method for the identification and isolation of terminator molecules causing enhanced expression of heterologous nucleic acid molecules to which the terminator molecules are functionally linked comprising the steps of identification of nucleic acid molecules comprising a sequence comprising in not more than 500 bp, preferably 400 bp, more preferable 300 bp most preferable 250 bp as defined in SEQ ID NO:1 or SEQ ID NO:2. In another step of the invention at hand, the respective nucleic acid molecules are isolated from their natural background such as genomic DNA with methods known to the person skilled in the art or synthesized. The nucleic acid molecules isolated from their natural background are then functionally coupled within an expression construct with heterologous nucleic acid molecules.

A further embodiment of the invention is a method for the identification and isolation of a terminator molecule causing enhanced expression of a heterologous nucleic acid molecule to which the terminator molecule is functionally linked within an expression construct, the method comprising
a) identifying a 3′UTR;
b) identifying a corresponding genomic DNA;
c) identifying a molecule from the group identified in b)
   i) that contains a fragment of 100 or more consecutive bases having 65% or more of the expression enhancing activity of the corresponding nucleic acid molecule having the sequence of SEQ ID NO: 1 or SEQ ID NO: 2; and
   ii) that comprises a sequence
      1) having at least 80% sequence identity to SEQ ID NO: 1 or SEQ ID NO: 2, or
      2) which hybridizes to the complement of SEQ ID NO: 1 or SEQ ID NO: 2 under hybridizing conditions equivalent to hybridization in 7% sodium dodecyl sulfate (SDS) at 50° C. with one or more washes in 2×SSC, 0.1% SDS at 50° C., conditions may also include hybridization at 7% sodium dodecyl sulfate (SDS), 0.5 M NaPO4, 1 mM EDTA at 50° C. with washing in 2×SSC, 0.1% SDS at 50° C. or 65° C.; and
d) isolating at least 100 bp of genomic DNA from b) or c).

The 3′UTRs and the corresponding genomic DNA may be identified by any method known to a person skilled in the art, for example sequence determination of full-length cDNAs, computational predictions for example based on the prediction of coding sequences in genomic DNA sequences or the use of annotated databases for e.g. cDNAs or genomic DNA.

The term “corresponding genomic DNA” is to be understood as genomic DNA, preferably plant genomic DNA, more preferably monocotyledonous plant genomic DNA comprising the sequence identified as 3′UTR or transcribed region in step A). The corresponding genomic DNA may comprise an identical sequence or a sequence having an identity of at least 60% or, preferably 70% or more, for example 80% or more, more preferably, the identity is 85% or more, more preferably the identity is 90% or more, even more preferably, the identity is 95% or more, 96% or more, 97% or more, 98% or more, in the most preferred embodiment, the identity is 99% or more to any of the sequences of the 3′UTR or transcribed region identified in step A).

Identification of molecules in the group identified in B) may for example be done with tools known to the skilled person, such as MatInspector of Genomatix Software GmbH, The MEME suite of the University of Queensland and University of Washington, or comparable tools. The isolation of the at least 250 bp, for example 300 bp, preferably at least 350 bp, for example 400 bp, more preferably at least 450 bp, for example 500 bp may be done with recombinant methods known in the art such as PCR, restriction cloning or gene synthesis. The isolated expression enhancing terminator molecules may in subsequent steps be functionally linked to promoters and/or nucleic acid molecules to be expressed. A skilled person is aware of various methods for functionally linking two or more nucleic acid molecules. Such methods may encompass restriction/ligation, ligase independent cloning, recombineering, recombination or synthesis. Other methods may be employed to functionally link two or more nucleic acid molecules.

A further embodiment of the invention is a method for producing an expression construct, which when expressed in a plant cell or plant or part thereof enhances expression of one or more nucleic acid molecule as compared to a corresponding control plant or part thereof, the method comprising
a) introducing a terminator molecule comprising one or more terminator molecules as defined in a) to e) at page 3 above;
and
b) functionally linking said one or more terminator molecule to a promoter and to a nucleic acid molecule being under the control of said promoter, wherein the terminator molecule is heterologous to said nucleic acid molecule and/or promoter.

Another embodiment of the invention is a method for producing a plant cell, plant or part thereof with enhanced expression of one or more nucleic acid molecule as compared to a corresponding control plant or part thereof, the method comprising
a) introducing one or more terminator molecule comprising the terminator molecule as defined in a) to e) at page 3 above into a plant cell, plant or part thereof;
b) integrating said one or more terminator into the genome of said plant cell, plant or part thereof, whereby said one or more terminator is functionally linked to an endogenous nucleic acid heterologous to said one or more terminator; and optionally
c) regenerating a plant or part thereof comprising said one or more terminator from said transformed cell.

The one or more expression enhancing terminator molecule may be introduced into the plant or part thereof by means of particle bombardment, protoplast electroporation, virus infection, Agrobacterium mediated transformation or any other approach known in the art. The expression enhancing terminator molecule may be introduced integrated for example into a plasmid or viral DNA or viral RNA. The expression enhancing terminator molecule may also be comprised on a BAC, YAC or artificial chromosome prior to introduction into the plant or part of the plant. It may be also introduced as a linear nucleic acid molecule comprising the expression enhancing terminator sequence wherein additional sequences may be present adjacent to the expression enhancing terminator sequence on the nucleic acid molecule. These sequences neighboring the expression enhancing terminator sequence may be from about 20 bp, for example 20 bp to several hundred base pairs, for example 100 bp or more and may facilitate integration into the genome for example by homologous recombination. Any other method for genome integration may be employed, for example targeted integration approaches, such as homologous recombination or random integration approaches, such as illegitimate recombination.

The endogenous nucleic acid to which the expression enhancing terminator molecule may be functionally linked may be any nucleic acid molecule. The nucleic acid molecule may be a protein coding nucleic acid molecule or a non-coding molecule such as antisense RNA, rRNA, tRNA, miRNA, ta-siRNA, siRNA, dsRNA, snRNA, snoRNA or any other non-coding RNA known in the art.

Another embodiment of the invention is a method for producing a plant cell, plant or part thereof with enhanced expression of one or more nucleic acid molecule as compared to a corresponding control plant or part thereof, the method comprising
a) providing an expression construct comprising one or more expression enhancing terminator molecule comprising the terminator molecule as defined in a) to e) at page 3 above, functionally linked to a promoter and to one or more nucleic acid molecule the latter being heterologous to said one or more terminator molecule and which is under the control of said promoter; and
b) integrating said expression construct comprising said one or more terminator molecule into the genome of said plant or part thereof and optionally
c) regenerating a plant or part thereof comprising said one or more expression construct from said transformed plant or part thereof.

The expression enhancing terminator molecule may be heterologous to the nucleic acid molecule which is under the control of said promoter to which the expression enhancing terminator is functionally linked or it may be heterologous to both the promoter and the nucleic acid molecule under the control of said promoter.

The one or more expression enhancing terminator molecule may be introduced into the plant or part thereof by means of particle bombardment, protoplast electroporation, virus infection, Agrobacterium mediated transformation or any other approach known in the art. The expression enhancing terminator molecule may be introduced integrated for example into a plasmid or viral DNA or viral RNA. The expression enhancing terminator molecule may also be comprised on a BAC, YAC or artificial chromosome prior to introduction into the plant or part of the plant. It may be also introduced as a linear nucleic acid molecule comprising the expression enhancing terminator sequence wherein additional sequences may be present adjacent to the expression enhancing terminator sequence on the nucleic acid molecule. These sequences neighboring the expression enhancing terminator sequence may be from about 20 bp, for example 20 bp to several hundred base pairs, for example 100 bp or more and may facilitate integration into the genome for example by homologous recombination. Any other method for genome integration may be employed, for example targeted integration approaches, such as homologous recombination or random integration approaches, such as illegitimate recombination.

The methods of the invention may be applied to any plant, for example gymnosperm or angiosperm, preferably angiosperm, for example dicotyledonous (dicot) or monocotyledonous (monocot) plants. Preferred monocotyledonous plants are for example corn, wheat, rice, barley, sorghum, musa, sugarcane, miscanthus and brachypodium, especially preferred monocotyledonous plants are corn, wheat and rice. Preferred dicotyledonous plants are for example soy, rape seed, canola, linseed, cotton, potato, sugar beet, tagetes and Arabidopsis, especially preferred dicotyledonous plants are soy, rape seed, canola and potato.

A plant exhibiting enhanced expression of a nucleic acid molecule as meant herein means a plant having a higher, preferably statistically significant higher expression of a nucleic acid molecule compared to a control plant grown under the same conditions without the respective expression enhancing terminator functionally linked to the respective nucleic acid molecule. Such control plant may be a wild-type plant or a transgenic plant comprising the same promoter controlling the same gene as in the plant of the invention wherein the promoter and/or the nucleic acid to be expressed is not linked to an expression enhancing terminator of the invention.

A recombinant expression construct comprising one or more expression enhancing terminator molecule comprising an expression enhancing terminator molecule as defined above at page 3 under a) to e) is a further embodiment of the invention. The recombinant expression construct may further comprise one or more promoter and optionally one or more nucleic acid molecule to which the one or more expression enhancing terminator molecule is functionally linked, at least the latter being heterologous to said one or more expression enhancing terminator molecule.

The expression enhancing terminator molecule may be heterologous to the nucleic acid molecule which is under the control of said promoter to which the expression enhancing terminator molecule is functionally linked or it may be heterologous to both the promoter and the nucleic acid molecule under the control of said promoter.

The expression construct may comprise one ore more, for example two or more, for example 5 or more, such as 10 or more combinations of promoters functionally linked to a expression enhancing terminator molecule and a nucleic acid molecule to be expressed which is heterologous to the respective expression enhancing terminator molecule. The expression construct may also comprise further expression constructs not comprising an expression enhancing terminator molecule.

A recombinant expression vector comprising one or more recombinant expression construct as defined above is another embodiment of the invention. A multitude of expression vectors that may be used in the present invention are known to a skilled person. Methods for introducing such a vector comprising such an expression construct comprising for example a promoter functionally linked to a expression enhancing terminator and optionally other elements such as a promoters, UTRs, NEENAs and the like into the genome of a plant and for recovering transgenic plants from a transformed cell are also well known in the art. Depending on the method used for the transformation of a plant or part thereof the entire vector might be integrated into the genome of said plant or part thereof or certain components of the vector might be integrated into the genome, such as, for example a T-DNA.

A transgenic cell or transgenic plant or part thereof comprising a recombinant expression vector as defined above or a recombinant expression construct as defined above is a further embodiment of the invention. The transgenic cell, transgenic plant or part thereof may be selected from the group consisting of bacteria, fungi, yeasts, or plant, insect or mammalian cells or plants. Preferred transgenic cells are bacteria, fungi, yeasts and plant cells. Preferred bacteria are Enterobacteria such as E. coli and bacteria of the genus Agrobacteria, for example Agrobacterium tumefeciens and Agrobacterium rhizogenes. Preferred plants are monocotyledonous or dicotyledonous plants for example monocotyledonous or dicotyledonous crop plants such as corn, soy, canola, cotton, potato, sugar beet, rice, wheat, sorghum, barley, musa, sugarcane, miscanthus and the like. Preferred crop plants are corn, rice, wheat, soy, canola, cotton or potato. Especially preferred dicotyledonous crop plants are soy, canola, cotton or potato. Especially preferred monocotyledonous crop plants are corn, wheat and rice.

A transgenic cell culture, transgenic seed, parts or propagation material derived from a transgenic cell or plant or part thereof as defined above comprising said heterologous expression enhancing terminator molecule comprising an expression enhancing terminator molecule as defined above at page 3 under a) to ) or said recombinant expression construct or said recombinant vector as defined above are other embodiments of the invention.

Transgenic parts or propagation material as meant herein comprise all tissues and organs, for example leaf, stem and fruit as well as material that is useful for propagation and/or regeneration of plants such as cuttings, scions, layers, branches or shoots comprising the respective expression enhancing terminator molecule, recombinant expression construct or recombinant vector.

A further embodiment of the invention is the use of the expression enhancing terminator molecule comprising an expression enhancing terminator molecule as defined above at page 3 under a) to e) or the recombinant construct or recombinant vector as defined above for enhancing expression in plants or parts thereof.

A further embodiment of the invention is a method for the production of an agricultural product by introducing an expression enhancing terminator molecule comprising an expression enhancing terminator molecule as defined above at page 3under a) to e) or the recombinant construct or recombinant vector as defined above into a plant, growing the plant, harvesting and processing the plant or parts thereof.

### DEFINITIONS

### Abbreviations

NEENA—nucleic acid expression enhancing nucleic acid, GFP—green fluorescence protein, GUS—beta-Glucuronidase, BAP—6-benzylaminopurine; 2,4-D—2,4-dichlorophenoxyacetic acid; MS—Murashige and Skoog medium; NAA—1-naphtaleneacetic acid; MES, 2-(N-morpholino-ethanesulfonic acid, IAA indole acetic acid; Kan: Kanamycin sulfate; GA3—Gibberellic acid; Timentin™: ticarcillin disodium/clavulanate potassium, microl: Microliter.

It is to be understood that this invention is not limited to the particular methodology or protocols. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

Agricultural product: The term “Agricultural product” as used in this application means any harvestable product from a plant. The plant products may be, but are not limited to, foodstuff, feedstuff, food supplement, feed supplement, fiber, cosmetic or pharmaceutical product. Foodstuffs are regarded as compositions used for nutrition or for supplementing nutrition. Animal feedstuffs and animal feed supplements, in particular, are regarded as foodstuffs. Agricultural products may as an example be plant extracts, proteins, amino acids, carbohydrates, fats, oils, polymers such as starch or fibers, vitamins, secondary plant products and the like.

Antiparallel: “Antiparallel” refers herein to two nucleotide sequences paired through hydrogen bonds between complementary base residues with phosphodiester bonds running in the 5′-3′ direction in one nucleotide sequence and in the 3′-5′ direction in the other nucleotide sequence.

Antisense: The term “antisense” refers to a nucleotide sequence that is inverted relative to its normal orientation for transcription or function and so expresses an RNA transcript that is complementary to a target gene mRNA molecule expressed within the host cell (e.g., it can hybridize to the target gene mRNA molecule or single stranded genomic DNA through Watson-Crick base pairing) or that is complementary to a target DNA molecule such as, for example genomic DNA present in the host cell.

Coding region: As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA).

In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

Double-stranded RNA: A “double-stranded RNA” molecule or “dsRNA” molecule comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule.

Endogenous: An “endogenous” nucleotide sequence refers to a nucleotide sequence, which is present in the genome of the untransformed plant cell.

Enhanced expression: “enhance” or “increase” the expression of a nucleic acid molecule in a plant cell are used equivalently herein and mean that the level of expression of the nucleic acid molecule in a plant, part of a plant or plant cell after applying a method of the present invention is higher than its expression in the plant, part of the plant or plant cell before applying the method, or compared to a reference plant lacking a recombinant nucleic acid molecule of the invention. For example, the reference plant is comprising the same construct which is only lacking the respective enhancing terminator of the invention. The term “enhanced” or “increased” as used herein are synonymous and means herein higher, preferably significantly higher expression of the nucleic acid molecule to be expressed. As used herein, an “enhancement” or “increase” of the level of an agent such as a protein, mRNA or RNA means that the level is increased relative to a substantially identical plant, part of a plant or plant cell grown under substantially identical conditions, lacking a recombinant nucleic acid molecule of the invention, for example lacking the enhancing terminator of the invention molecule, the recombinant construct or recombinant vector of the invention. As used herein, “enhancement” or “increase” of the level of an agent, such as for example a preRNA, mRNA, rRNA, tRNA, snoRNA, snRNA expressed by the target gene and/or of the protein product encoded by it, means that the level is increased 20% or more, for example 50% or more, preferably 100% or more, more preferably 3 fold or more, even more preferably 15 fold or more, most preferably 10 fold or more for example 20 fold relative to a cell or organism lacking a recombinant nucleic acid molecule of the invention. The enhancement or increase can be determined by methods with which the skilled worker is familiar. Thus, the enhancement or increase of the nucleic acid or protein quantity can be determined for example by an immunological detection of the protein. Moreover, techniques such as protein assay, fluorescence, Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a plant or plant cell. Depending on the type of the induced protein product, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254). As one example for quantifying the activity of a protein, the detection of luciferase activity is described in the Examples below.

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Expression construct: “Expression construct” as used herein mean a DNA sequence capable of directing expression of a particular nucleotide sequence in an appropriate part of a plant or plant cell, comprising a promoter functional in said part of a plant or plant cell into which it will be introduced, operatively linked to the nucleotide sequence of interest which is—optionally—operatively linked to termination signals. If translation is required, it also typically comprises sequences required for proper translation of the nucleotide sequence. The coding region may code for a protein of interest but may also code for a functional RNA of interest, for example RNAa, siRNA, snoRNA, snRNA, microRNA, ta-siRNA or any other noncoding regulatory RNA, in the sense or antisense direction. The expression construct comprising the nucleotide sequence of interest may be chimeric, meaning that one or more of its components is heterologous with respect to one or more of its other components. The expression construct may also be one, which is naturally occurring but has been obtained in a recombinant form useful for heterologous expression. Typically, however, the expression construct is heterologous with respect to the host, i.e., the particular DNA sequence of the expression construct does not occur naturally in the host cell and must have been introduced into the host cell or an ancestor of the host cell by a transformation event. The expression of the nucleotide sequence in the expression construct may be under the control of a seed-specific and/or seed-preferential promoter or of an inducible promoter, which initiates transcription only when the host cell is exposed to some particular external stimulus. In the case of a plant, the promoter can also be specific to a particular tissue or organ or stage of development.

Foreign: The term “foreign” refers to any nucleic acid molecule (e.g., gene sequence) which is introduced into the genome of a cell by experimental manipulations and may include sequences found in that cell so long as the introduced sequence contains some modification (e.g., a point mutation, the presence of a selectable marker gene, etc.) and is therefore distinct relative to the naturally-occurring sequence.

Functional linkage: The term “functional linkage” or “functionally linked” is to be understood as meaning, for example, the sequential arrangement of a regulatory element (e.g. a promoter) with a nucleic acid sequence to be expressed and, if appropriate, further regulatory elements (such as e.g., a terminator or a NEENA) in such a way that each of the regulatory elements can fulfill its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (N.Y.); Silhavy et al. (1984) Experiments with Gene Fusions, Cold Spring Harbor Laboratory, Cold Spring Harbor (N.Y.); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and be inserted into a plant genome, for example by transformation.

Gene: The term “gene” refers to a region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

Genome and genomic DNA: The terms “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

Heterologous: The term “heterologous” with respect to a nucleic acid molecule or DNA refers to a nucleic acid molecule which is operably linked to, or is manipulated to become operably linked to, a second nucleic acid molecule to which it is not operably linked in nature, or to which it is operably linked at a different location in nature. A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

Hybridization: The term “hybridization” as used herein includes “any process by which a strand of nucleic acid molecule joins with a complementary strand through base pairing.” (J. Coombs (1994) Dictionary of Biotechnology, Stockton Press, New York). Hybridization and the strength of hybridization (i.e., the strength of the association between the nucleic acid molecules) is impacted by such factors as the degree of complementarity between the nucleic acid molecules, stringency of the conditions involved, the Tm of the formed hybrid, and the G:C ratio within the nucleic acid molecules. As used herein, the term “Tm” is used in reference to the “melting temperature.” The melting temperature is the temperature at which a population of double-stranded nucleic acid molecules becomes half dissociated into single strands. The equation for calculating the Tm of nucleic acid molecules is well known in the art. As indicated by standard references, a simple estimate of the Tm value may be calculated by the equation: Tm=81.5+0.41(% G+C), when a nucleic acid molecule is in aqueous solution at 1 M NaCl [see e.g., Anderson and Young, Quantitative Filter Hybridization, in Nucleic Acid Hybridization (1985)]. Other references include more sophisticated computations, which take structural as well as sequence characteristics into account for the calculation of Tm. Stringent conditions, are known to those skilled in the art and can be found in Current Protocols in Molecular Biology, John Wiley & Sons, N.Y. (1989), 6.3.1-6.3.6.

“Identity”: “Identity” when used in respect to the comparison of two or more nucleic acid or amino acid molecules means that the sequences of said molecules share a certain degree of sequence similarity, the sequences being partially identical.

To determine the percentage identity (homology is herein used interchangeably) of two amino acid sequences or of two nucleic acid molecules, the sequences are written one underneath the other for an optimal comparison (for example gaps may be inserted into the sequence of a protein or of a nucleic acid in order to generate an optimal alignment with the other protein or the other nucleic acid).

The amino acid residues or nucleic acid molecules at the corresponding amino acid positions or nucleotide positions are then compared. If a position in one sequence is occupied by the same amino acid residue or the same nucleic acid molecule as the corresponding position in the other sequence, the molecules are homologous at this position (i.e. amino acid or nucleic acid “homology” as used in the present context corresponds to amino acid or nucleic acid “identity”. The percentage homology between the two sequences is a function of the number of identical positions shared by the sequences (i.e. % homology=number of identical positions/total number of positions×100). The terms “homology” and “identity” are thus to be considered as synonyms.

For the determination of the percentage identity of two or more amino acids or of two or more nucleotide sequences several computer software programs have been developed. The identity of two or more sequences can be calculated with for example the software fasta, which presently has been used in the version fasta 3 (W. R. Pearson and D. J. Lipman, PNAS 85, 2444 (1988); W. R. Pearson, Methods in Enzymology 183, 63 (1990); W. R. Pearson and D. J. Lipman, PNAS 85, 2444 (1988); W. R. Pearson, Enzymology 183, 63 (1990)). Another useful program for the calculation of identities of different sequences is the standard blast program, which is included in the Biomax pedant software (Biomax, Munich, Federal Republic of Germany). This leads unfortunately sometimes to suboptimal results since BLAST does not always include complete sequences of the subject and the query. Nevertheless as this program is very efficient it can be used for the comparison of a huge number of sequences. The following settings are typically used for such a comparison of sequences:

-p Program Name [String]; -d Database [String]; default=nr; -i Query File [File In]; default=stdin; -e Expectation value (E) [Real]; default=10.0; -m alignment view options: 0=pairwise; 1=query-anchored showing identities; 2=query-anchored no identities; 3=flat query-anchored, show identities; 4=flat query-anchored, no identities; 5=query-anchored no identities and blunt ends; 6=flat query-anchored, no identities and blunt ends; 7=XML Blast output; 8=tabular; 9 tabular with comment lines [Integer]; default=0; -o BLAST report Output File [File Out] Optional; default=stdout; -F Filter query sequence (DUST with blastn, SEG with others) [String]; default=T; -G Cost to open a gap (zero invokes default behavior) [Integer]; default=0; -E Cost to extend a gap (zero invokes default behavior) [Integer]; default=0; -X X dropoff value for gapped alignment (in bits) (zero invokes default behavior); blastn 30, megablast 20, tblastx 0, all others 15 [Integer]; default=0; -l Show GI's in defines [T/F]; default=F; -q Penalty for a nucleotide mismatch (blastn only) [Integer]; default=−3; -r Reward for a nucleotide match (blastn only) [Integer]; default=1; -v Number of database sequences to show one-line descriptions for (V) [Integer]; default=500; -b Number of database sequence to show alignments for (B) [Integer]; default=250; -f Threshold for extending hits, default if zero; blastp 11, blastn 0, blastx 12, tblastn 13; tblastx 13, megablast 0 [Integer]; default=0; -g Perform gapped alignment (not available with tblastx) [T/F]; default=T; -Q Query Genetic code to use [Integer]; default=1; -D DB Genetic code (for tblast[nx] only) [Integer]; default=1; -a Number of processors to use [Integer]; default=1; -O SeqAlign file [File Out] Optional; -J Believe the query define [T/F]; default=F; -M Matrix [String]; default=BLOSUM62; -W Word size, default if zero (blastn 11, megablast 28, all others 3) [Integer]; default=0; -z Effective length of the database (use zero for the real size) [Real]; default=0; -K Number of best hits from a region to keep (off by default, if used a value of 100 is recommended) [Integer]; default=0; -P 0 for multiple hit, 1 for single hit [Integer]; default=0; -Y Effective length of the search space (use zero for the real size) [Real]; default=0; -S Query strands to search against database (for blast[nx], and tblastx); 3 is both, 1 is top, 2 is bottom [Integer]; default=3; -T Produce HTML output [T/F]; default=F; -l Restrict search of database to list of GI's [String] Optional; -U Use lower case filtering of FASTA sequence [T/F] Optional; default=F; -y X dropoff value for ungapped extensions in bits (0.0 invokes default behavior); blastn 20, megablast 10, all others 7 [Real]; default=0.0; -Z X dropoff value for final gapped alignment in bits (0.0 invokes default behavior); blastn/megablast 50, tblastx 0, all others 25 [Integer]; default=0; -R PSI-TBLASTN checkpoint file [File In] Optional; -n MegaBlast search [T/F]; default=F; -L Location on query sequence [String] Optional; -A Multiple Hits window size, default if zero (blastn/megablast 0, all others 40 [Integer]; default=0; -w Frame shift penalty (OOF algorithm for blastx) [Integer]; default=0; -t Length of the largest intron allowed in tblastn for linking HSPs (0 disables linking) [Integer]; default=0.

Results of high quality are reached by using the algorithm of Needleman and Wunsch or Smith and Waterman. Therefore programs based on said algorithms are preferred. Advantageously the comparisons of sequences can be done with the program PileUp (J. Mol. Evolution., 25, 351 (1987), Higgins et al., CABIOS 5, 151 (1989)) or preferably with the programs “Gap” and “Needle”, which are both based on the algorithms of Needleman and Wunsch (J. Mol. Biol. 48; 443 (1970)), and “BestFit”, which is based on the algorithm of Smith and Waterman (Adv. Appl. Math. 2; 482 (1981)). “Gap” and “BestFit” are part of the GCG software-package (Genetics Computer Group, 575 Science Drive, Madison, Wis., USA 53711 (1991); Altschul et al., (Nucleic Acids Res. 25, 3389 (1997)), “Needle” is part of The European Molecular Biology Open Software Suite (EMBOSS) (Trends in Genetics 16 (6), 276 (2000)). Therefore preferably the calculations to determine the percentages of sequence homology are done with the programs “Gap” or “Needle” over the whole range of the sequences. The following standard adjustments for the comparison of nucleic acid sequences were used for “Needle”: matrix: EDNAFULL, Gap_penalty: 10.0, Extend_penalty: 0.5. The following standard adjustments for the comparison of nucleic acid sequences were used for “Gap”: gap weight: 50, length weight: 3, average match: 10.000, average mismatch: 0.000.

For example a sequence, which is said to have 80% identity with sequence SEQ ID NO: 1 at the nucleic acid level is understood as meaning a sequence which, upon comparison with the sequence represented by SEQ ID NO: 1 by the above program “Needle” with the above parameter set, has a 80% identity. Preferably the homology is calculated on the complete length of the query sequence, for example SEQ ID NO: 1.

Intron: refers to sections of DNA (intervening sequences) within a gene that do not encode part of the protein that the gene produces, and that is spliced out of the mRNA that is transcribed from the gene before it is exported from the cell nucleus. Intron sequence refers to the nucleic acid sequence of an intron. Thus, introns are those regions of DNA sequences that are transcribed along with the coding sequence (exons) but are removed during the formation of mature mRNA. Introns can be positioned within the actual coding region or in either the 5′ or 3′ untranslated leaders of the pre-mRNA (unspliced mRNA). Introns in the primary transcript are excised and the coding sequences are simultaneously and precisely ligated to form the mature mRNA. The junctions of introns and exons form the splice site. The sequence of an intron begins with GU and ends with AG. Furthermore, in plants, two examples of AU-AC introns have been described: the fourteenth intron of the RecA-like protein gene and the seventh intron of the G5 gene from Arabidopsis thaliana are AT-AC introns. Pre-mRNAs containing introns have three short sequences that are—beside other sequences—essential for the intron to be accurately spliced. These sequences are the 5′splice-site, the 3′splice-site, and the branchpoint. mRNA splicing is the removal of intervening sequences (introns) present in primary mRNA transcripts and joining or ligation of exon sequences. This is also known as cis-splicing which joins two exons on the same RNA with the removal of the intervening sequence (intron). The functional elements of an intron is comprising sequences that are recognized and bound by the specific protein components of the spliceosome (e.g. splicing consensus sequences at the ends of introns). The interaction of the functional elements with the spliceosome results in the removal of the intron sequence from the premature mRNA and the rejoining of the exon sequences. Introns have three short sequences that are essential—although not sufficient—for the intron to be accurately spliced. These sequences are the 5′ splice site, the 3′ splice site and the branch point. The branchpoint sequence is important in splicing and splice-site selection in plants. The branchpoint sequence is usually located 10-60 nucleotides upstream of the 3′ splice site.

Isogenic: organisms (e.g., plants), which are genetically identical, except that they may differ by the presence or absence of a heterologous DNA sequence.

Isolated: The term “isolated” or “isolation” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring polynucleotide or polypeptide present in a living plant is not isolated, but the same polynucleotide or polypeptide, separated from some or all of the coexisting materials in the natural system, is isolated. Such polynucleotides can be part of a vector and/or such polynucleotides or polypeptides could be part of a composition, and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 1 includes, by way of example, such nucleic acid sequences in cells which ordinarily contain SEQ ID NO:1 where the nucleic acid sequence is in a chromosomal or extrachromosomal location different from that of natural cells, or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Minimal Promoter: promoter elements, particularly a TATA element, that are inactive or that have greatly reduced promoter activity in the absence of upstream activation. In the presence of a suitable transcription factor, the minimal promoter functions to permit transcription.

NEENA: see “Nucleic acid expression enhancing nucleic acid”.

Non-coding: The term “non-coding” refers to sequences of nucleic acid molecules that do not encode part or all of an expressed protein. Non-coding sequences include but are not limited to introns, enhancers, promoter regions, 3′ untranslated regions, and 5′ untranslated regions.

Nucleic acid expression enhancing nucleic acid (NEENA): The term “nucleic acid expression enhancing nucleic acid” refers to a sequence and/or a nucleic acid molecule of a specific sequence having the intrinsic property to enhance expression of a nucleic acid under the control of a promoter to which the NEENA is functionally linked. Unlike promoter sequences, the NEENA as such is not able to drive expression. In order to fulfill the function of enhancing expression of a nucleic acid molecule functionally linked to the NEENA, the NEENA itself has to be functionally linked to a promoter. In distinction to enhancer sequences known in the art, the NEENA is acting in cis but not in trans and has to be located close to the transcription start site of the nucleic acid to be expressed.

Nucleic acids and nucleotides: The terms “Nucleic Acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof, in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used inter-changeably herein with “gene”, “cDNA, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Plant: is generally understood as meaning any eukaryotic single- or multi-celled organism or a cell, tissue, organ, part or propagation material (such as seeds or fruit) of same which is capable of photosynthesis. Included for the purpose of the invention are all genera and species of higher and lower plants of the Plant Kingdom. Annual, perennial, monocotyledonous and dicotyledonous plants are preferred. The term includes the mature plants, seed, shoots and seedlings and their derived parts, propagation material (such as seeds or microspores), plant organs, tissue, protoplasts, callus and other cultures, for example cell cultures, and any other type of plant cell grouping to give functional or structural units. Mature plants refer to plants at any desired developmental stage beyond that of the seedling. Seedling refers to a young immature plant at an early developmental stage. Annual, biennial, monocotyledonous and dicotyledonous plants are preferred host organisms for the generation of transgenic plants. The expression of genes is furthermore advantageous in all ornamental plants, useful or ornamental trees, flowers, cut flowers, shrubs or lawns. Plants which may be mentioned by way of example but not by limitation are angiosperms, bryophytes such as, for example, Hepaticae (liverworts) and Musci (mosses); Pteridophytes such as ferns, horsetail and club mosses; gymnosperms such as conifers, cycads, ginkgo and Gnetatae; algae such as Chlorophyceae, Phaeophpyceae, Rhodophyceae, Myxophyceae, Xanthophyceae, Bacillariophyceae (diatoms), and Euglenophyceae. Preferred are plants which are used for food or feed purpose such as the families of the Leguminosae such as pea, alfalfa and soya; Gramineae such as rice, maize, wheat, barley, sorghum, millet, rye, triticale, or oats; the family of the Umbelliferae, especially the genus Daucus, very especially the species carota (carrot) and Apium, very especially the species Graveolens dulce (celery) and many others; the family of the Solanaceae, especially the genus Lycopersicon, very especially the species esculentum (tomato) and the genus Solanum, very especially the species tuberosum (potato) and melongena (egg plant), and many others (such as tobacco); and the genus Capsicum, very especially the species annuum (peppers) and many others; the family of the Leguminosae, especially the genus Glycine, very especially the species max (soybean), alfalfa, pea, lucerne, beans or peanut and many others; and the family of the Cruciferae (Brassicacae), especially the genus Brassica, very especially the species napus (oil seed rape), campestris (beet), oleracea cv Tastie (cabbage), oleracea cv Snowball Y (cauliflower) and oleracea cv Emperor (broccoli); and of the genus Arabidopsis, very especially the species thaliana and many others; the family of the Compositae, especially the genus Lactuca, very especially the species sativa (lettuce) and many others; the family of the Asteraceae such as sunflower, Tagetes, lettuce or Calendula and many other; the family of the Cucurbitaceae such as melon, pumpkin/squash or zucchini, and linseed. Further preferred are cotton, sugar cane, hemp, flax, chillies, and the various tree, nut and wine species.

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Pre-protein: Protein, which is normally targeted to a cellular organelle, such as a chloroplast, and still comprising its transit peptide.

Primary transcript: The term “primary transcript” as used herein refers to a premature RNA transcript of a gene. A “primary transcript” for example still comprises introns and/or is not yet comprising a polyA tail or a cap structure and/or is missing other modifications necessary for its correct function as transcript such as for example trimming or editing.

Promoter: The terms “promoter”, or “promoter sequence” are equivalents and as used herein, refer to a DNA sequence which when ligated to a nucleotide sequence of interest is capable of controlling the transcription of the nucleotide sequence of interest into RNA. Such promoters can for example be found in the following public databases http://www.grassius.org/grasspromdb.html, http://mendel.cs.rhul.ac.uk/mendel.php?topic=plantprom, http://ppdb.gene.nagoya-u.ac.jp/cgi-bin/index.cgi. Promoters listed there may be addressed with the methods of the invention and are herewith included by reference. A promoter is located 5′ (i.e., upstream), proximal to the transcriptional start site of a nucleotide sequence of interest whose transcription into mRNA it controls, and provides a site for specific binding by RNA polymerase and other transcription factors for initiation of transcription. Said promoter comprises for example the at least 10 kb, for example 5 kb or 2 kb proximal to the transcription start site. It may also comprise the at least 1500 bp proximal to the transcriptional start site, preferably the at least 1000 bp, more preferably the at least 500 bp, even more preferably the at least 400 bp, the at least 300 bp, the at least 200 bp or the at least 100 bp. In a further preferred embodiment, the promoter comprises the at least 50 bp proximal to the transcription start site, for example, at least 25 bp. The promoter does not comprise exon and/or intron regions or 5′ untranslated regions. The promoter may for example be heterologous or homologous to the respective plant. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur or from pathogens for this host cells (e.g., plants or plant pathogens like plant viruses). A plant specific promoter is a promoter suitable for regulating expression in a plant. It may be derived from a plant but also from plant pathogens or it might be a synthetic promoter designed by man. If a promoter is an inducible promoter, then the rate of transcription increases in response to an inducing agent. Also, the promoter may be regulated in a tissue-specific or tissue preferred manner such that it is only or predominantly active in transcribing the associated coding region in a specific tissue type(s) such as leaves, roots or meristem. The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter, which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., GUS activity staining, GFP protein or immunohistochemical staining. The term “constitutive” when made in reference to a promoter or the expression derived from a promoter means that the promoter is capable of directing transcription of an operably linked nucleic acid molecule in the absence of a stimulus (e.g., heat shock, chemicals, light, etc.) in the majority of plant tissues and cells throughout substantially the entire lifespan of a plant or part of a plant. Typically, constitutive promoters are capable of directing expression of a transgene in substantially any cell and any tissue.

Promoter specificity: The term “specificity” when referring to a promoter means the pattern of expression conferred by the respective promoter. The specificity describes the tissues and/or developmental status of a plant or part thereof, in which the promoter is conferring expression of the nucleic acid molecule under the control of the respective promoter. Specificity of a promoter may also comprise the environmental conditions, under which the promoter may be activated or down-regulated such as induction or repression by biological or environmental stresses such as cold, drought, wounding or infection.

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

Recombinant: The term “recombinant” with respect to nucleic acid molecules refers to nucleic acid molecules produced by recombinant DNA techniques. Recombinant nucleic acid molecules may also comprise molecules, which as such does not exist in nature but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecule” may also comprise a “recombinant construct” which comprises, preferably operably linked, a sequence of nucleic acid molecules not naturally occurring in that order. Preferred methods for producing said recombinant nucleic acid molecule may comprise cloning techniques, directed or non-directed mutagenesis, synthesis or recombination techniques.

“Seed-specific promoter” in the context of this invention means a promoter which is regulating transcription of a nucleic acid molecule under control of the respective promoter in seeds wherein the transcription in any tissue or cell of the seeds contribute to more than 90%, preferably more than 95%, more preferably more than 99% of the entire quantity of the RNA transcribed from said nucleic acid sequence in the entire plant during any of its developmental stage. The term “seed-specific expression” is to be understood accordingly.

“Seed-preferential promoter” in the context of this invention means a promoter which is regulating transcription of a nucleic acid molecule under control of the respective promoter in seeds wherein the transcription in any tissue or cell of the seeds contribute to more than 50%, preferably more than 70%, more preferably more than 80% of the entire quantity of the RNA transcribed from said nucleic acid sequence in the entire plant during any of its developmental stage. The term “seed-preferential expression” is to be understood accordingly.

Sense: The term “sense” is understood to mean a nucleic acid molecule having a sequence which is complementary or identical to a target sequence, for example a sequence which binds to a protein transcription factor and which is involved in the expression of a given gene. According to a preferred embodiment, the nucleic acid molecule comprises a gene of interest and elements allowing the expression of the said gene of interest.

Significant increase or decrease: An increase or decrease, for example in enzymatic activity or in gene expression, that is larger than the margin of error inherent in the measurement technique, preferably an increase or decrease by about 2-fold or greater of the activity of the control enzyme or expression in the control cell, more preferably an increase or decrease by about 5-fold or greater, and most preferably an increase or decrease by about 10-fold or greater.

Small nucleic acid molecules: “small nucleic acid molecules” are understood as molecules consisting of nucleic acids or derivatives thereof such as RNA or DNA. They may be double-stranded or single-stranded and are between about 15 and about 30 bp, for example between 15 and 30 bp, more preferred between about 19 and about 26 bp, for example between 19 and 26 bp, even more preferred between about 20 and about 25 bp for example between 20 and 25 bp. In a especially preferred embodiment the oligonucleotides are between about 21 and about 24 bp, for example between 21 and 24 bp. In a most preferred embodiment, the small nucleic acid molecules are about 21 bp and about 24 bp, for example 21 bp and 24 bp.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the later being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

Terminator: The term “terminator”, “transcription terminator”, or “transcription terminator sequence” as used herein is intended to mean a sequence located in the 3′UTR of a gene that causes a polymerase to stop forming phosphodiester bonds and release the nascent transcript. As used herein, the terminator comprises the 3′UTR structure necessary for efficient production of a messenger RNA. The terminator sequence leads to or initiates a stop of transcription of a nucleic acid sequence initiated from a promoter. Preferably, a transcription terminator sequence further comprises sequences which cause polyadenylation of the transcript. A transcription terminator may, for example, comprise one or more polyadenylation signal sequences, one or more polyadenylation attachment sequences, and downstream sequence of various lengths which causes termination of transcription. It has to be understood that also sequences downstream of sequences coding for the 3′-untranslated region of an expressed RNA transcript may be part of a transcription terminator although the sequence itself is not expressed as part of the RNA transcript. Furthermore, a transcription terminator may comprise additional sequences, which may influence its functionality, such a 3′-untranslated sequences (i.e. sequences of a gene following the stop-codon of the coding sequence). Transcription termination may involve various mechanisms including but not limited to induced dissociation of RNA polymerase II from their DNA template.

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with a recombinant DNA molecule that preferably comprises a suitable promoter operatively linked to a DNA sequence of interest.

Vector: As used herein, the term “vector” refers to a nucleic acid molecule capable of transporting another nucleic acid molecule to which it has been linked. One type of vector is a genomic integrated vector, or “integrated vector”, which can become integrated into the chromosomal DNA of the host cell. Another type of vector is an episomal vector, i.e., a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context. Expression vectors designed to produce RNAs as described herein in vitro or in vivo may contain sequences recognized by any RNA polymerase, including mitochondrial RNA polymerase, RNA pol I, RNA pol II, and RNA pol III. These vectors can be used to transcribe the desired RNA molecule in the cell according to this invention. A plant transformation vector is to be understood as a vector suitable in the process of plant transformation.

Wild-type: The term “wild-type”, “natural” or “natural origin” means with respect to an organism, polypeptide, or nucleic acid sequence, that said organism is naturally occurring or available in at least one naturally occurring organism which is not changed, mutated, or otherwise manipulated by man.

### EXAMPLES

The terminators from several genes were assayed in stable, transformed Zea mays seedlings. The terminator molecules, Terminator 1 and 2, encoded by SEQ ID NO:1 and SEQ ID NO:2, respectively, were operative and supported higher levels of reporter gene expression than the NOS terminator (SEQ ID NO:3).

Unless indicated otherwise, cloning procedures carried out for the purposes of the present invention including restriction digest, agarose gel electrophoresis, purification of nucleic acids, ligation of nucleic acids, transformation, selection and cultivation of bacterial cells were performed as described (Sambrook et al., 1989). Sequence analyses of recombinant DNA were performed with a laser fluorescence DNA sequencer (Applied Biosystems, Foster City, Calif., USA) using the Sanger technology (Sanger et al., 1977). Unless described otherwise, chemicals and reagents were obtained from Sigma Aldrich (Sigma Aldrich, St. Louis, USA), from Promega (Madison, Wis., USA), Duchefa (Haarlem, The Netherlands) or Invitrogen (Carlsbad, Calif., USA). Restriction endonucleases were from New England Biolabs (Ipswich, Mass., USA) or Roche Diagnostics GmbH (Penzberg, Germany).

Chemicals and Common Methods

GUS Histochemical Staining

GUS histochemical staining was performed as previously described (Jefferson et al., EMBO J. 6:3901-3907, 1987).

DsRed Fluorescence Scanning

Root tips (˜1 cm long) and leaf tissue (˜1 cm²) from 5-leaf stage T₀ seedlings were sampled and placed into the 24-well plates containing 0.5 ml GUS buffer to prevent dehydration. Prior to fluorescence analysis, the buffer was removed. DsRed analysis was performed on a Typhoon 9400 (Emission filter: 580 BP 30 cy3 TAMRA AlexaFluor 546, PMT=600, Laser=Green 532).

MUG Assay Experiment Protocol

MUG assays (Marker Gene Technologies, Product #M0877; Eugene, Oreg.) were performed as follows:
1. Foliar extract:
   1.1. Add enough ground leaf tissue up to the 100·L line of a microfuge tube;
   1.2. Add 500·L of cold GUS extraction buffer to each sample and vortex to mix;
   1.3. Incubate the sample at room temperature 10 minutes;
   1.4. Spin the extracts at 4° C. for 3 minutes.
2. Dilute the GUS Assay Buffer 20 fold. Vortex to mix well. Wrap foil around the tube to protect from light. The final concentration of the 4-MUG is 0.1 mM.
3. Dilute the Carbonate Stop Buffer 5 fold. Vortex to mix well. The final concentration of the Na₂CO₃ in the diluted stop solution is 0.2M.
4. Prepare a 96-well plate with extraction buffer, plant extract and assay buffer. Mix by pipetting. Cover plate with foil plate seal.
5. Incubate at 37° C. for 30 minutes.
6. After the 30 minute incubation, take the plate out of the 37° C. incubator and immediately add stop solution. Mix well. Cover plate with a foil seal.
7. Centrifuge the plate to eliminate air bubbles (˜2500-3000 rpm for 1-2 minutes).
8. Measure the fluorescence with emission at 465 nm and excitation at 360 nm immediately on Flex Station II.

Fluorescence units were calculated on a per unit mass protein (BCA Protein Assay Kit, Pierce Scientific; Rockford, Ill.).

Transcription Read-Through

Terminator leakage is defined as the relative ratio of transcript levels containing the GFP (an exogenous) target sequence (downstream of the terminator): transcript levels containing GUS sequence (upstream of the terminator). Transcript levels were determined using the Relative Quantification Method (ΔC(t)=C(t_{target})−C(t_{reference}). C(t), the threshold cycle, is defined as the number of cycles at which the fluorescence exceeds the threshold. ZmGPC1 was used as the internal reference gene.

### Example 1

### Terminator 1

To evaluate the influence of the various terminators, the terminator regions were fused to a 6-glucoronidase (GUS) gene under the control of the maize Ubiquitin promoter. For an internal expression control, the ZmUbi promoter also governed the expression of the DsRed reporter gene. The relative expression of the two reporter genes, from the same promoter, allowed for assessing any potential detrimental effects on transcription due to the terminator-of-interest. Fused immediately downstream of the terminator was a partial GFP encoding fragment. This exogenous sequence was used to determine the level of RNA transcription read-through via qPCR assays.

Terminators 1 and 2 were isolated from maize genes. Terminator 1 corresponds to Zea mays locus GRMZM2G305446, and is associated with the aquaporin transport protein. Terminator 2 corresponds to Zea mays locus GRMZM2G442555, and is associated with the core histone H2A/H2B/H3/H4 comain containing protein.

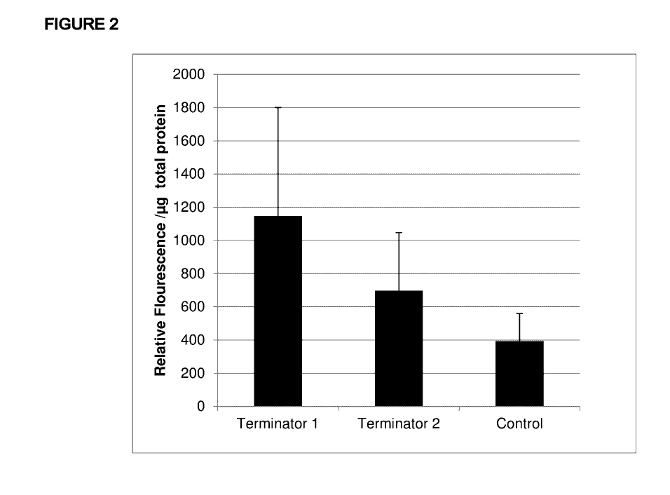

Transgenic seedlings comprising Terminator 1 comprising the nucleic acid sequence in SEQ ID NO:1 displayed weak-to-medium GUS staining. However, these plantlets had strong MUG fluorescence (see FIG. 2). MUG fluorescence was significantly different than the control seedlings using Terminator 3 comprising the nucleic acid sequence in SEQ ID NO:3. Terminator 1 exhibited higher levels of expression of the reporter gene compared to the other terminators tested.

Transcription read-through with Terminator 1 was significantly lower than that of the control plants (0.35±0.70 vs. 1.27±1.22%)

### Example 2

### Terminator 2

Seedlings comprising Terminator 2 comprising the nucleic acid sequence in SEQ ID NO:2 displayed medium-to-high GUS activity. MUG fluorescence was greater relative to the control plant comprising Terminator 3 (SEQ ID NO:3) (see FIG. 2).

1. A recombinant expression construct comprising a terminator molecule comprising at least one of SEQ ID NO: 1 or SEQ ID NO: 2, or the complement or reverse complement thereof, wherein said terminator molecule causes enhanced expression of one or more heterologous nucleic acid molecule to which the terminator molecule is functionally linked.

2. A recombinant expression construct comprising a terminator molecule comprising a nucleic acid sequence selected from the group consisting of
a) a nucleic acid molecule comprising the sequence of SEQ ID NO: 1 or SEQ ID NO: 2;
b) a nucleic acid molecule comprising a sequence having at least 80% sequence identity to SEQ ID NO: 1 or SEQ ID NO: 2;
c) a fragment of 100 or more consecutive bases of a nucleic acid molecule of a) or b) which has expression enhancing activity of the corresponding nucleic acid molecule having the sequence of SEQ ID NO: 1 or SEQ ID NO: 2;
d) a nucleic acid molecule which is the complement or reverse complement of the nucleic acid molecule of a) or b);
e) a nucleic acid molecule of 100 nucleotides or more, hybridizing under conditions equivalent to hybridization in 7% sodium dodecyl sulfate (SDS) at 50° C. with one or more washes in 2×SSC, 0.1% SDS at 50° C. to the complement of a nucleic acid molecule comprising at least 50 consecutive nucleotides of the transcription enhancing terminator of SEQ ID NO: 1 or SEQ ID NO: 2 or the complement thereof;
wherein the terminator molecule causes enhanced expression of one or more heterologous nucleic acid molecule to which the terminal molecule is functionally linked.

3. A method for the identification and isolation of a terminator molecule causing enhanced expression of a heterologous nucleic acid molecule to which the terminator molecule is functionally linked within an expression construct, the method comprising
a) identifying a 3′UTR;
b) identifying a corresponding genomic DNA;
c) identifying a molecule from the group identified in b)
i) that contains a fragment of 100 or more consecutive bases having 65% or more of the expression enhancing activity of the corresponding nucleic acid molecule having the sequence of SEQ ID NO: 1 or SEQ ID NO: 2; and
ii) that comprises a sequence
1) having at least 80% sequence identity to SEQ ID NO: 1 or SEQ ID NO: 2, or
2) which hybridizes to the complement of SEQ ID NO: 1 or SEQ ID NO: 2 under hybridizing conditions equivalent to hybridization in 7% sodium dodecyl sulfate (SDS) at 50° C. with one or more washes in 2×SSC, 0.1% SDS at 50° C.; and
d) isolating at least 100 bp of genomic DNA from b) or c).

4. A method for producing an expression construct, which when expressed in a plant cell or plant or part thereof enhances expression of one or more nucleic acid molecule as compared to a corresponding control plant or part thereof, the method comprising
a) introducing a terminator molecule comprising one or more terminator molecules as defined in claim 2 a) to e); and
b) functionally linking said one or more terminator molecule to a promoter and to a nucleic acid molecule being under the control of said promoter, wherein the terminator molecule is heterologous to said nucleic acid molecule and/or promoter.

5. A method for producing a plant cell, plant or part thereof with enhanced expression of one or more nucleic acid molecule as compared to a corresponding control plant or part thereof, the method comprising
a) introducing one or more terminator molecule comprising the terminator molecule as defined in claim 2 a) to e) into a plant cell, plant or part thereof;
b) integrating said one or more terminator into the genome of said plant cell, plant or part thereof, whereby said one or more terminator is functionally linked to an endogenous nucleic acid heterologous to said one or more terminator; and optionally
c) regenerating a plant or part thereof comprising said one or more terminator from said transformed cell.

6. A method for producing a plant cell, plant or part thereof with enhanced expression of one or more nucleic acid molecule as compared to a corresponding control plant or part thereof, the method comprising
a) providing an expression construct comprising one or more expression enhancing terminator molecule comprising the terminator molecule as defined in claim 2 a) to e), functionally linked to a promoter and to one or more nucleic acid molecule the latter being heterologous to said one or more terminator molecule and which is under the control of said promoter; and
b) integrating said expression construct comprising said one or more terminator molecule into the genome of said plant or part thereof and optionally
c) regenerating a plant or part thereof comprising said one or more expression construct from said transformed plant or part thereof.

7. The method of claim 5, wherein the plant is a monocot plant or a dicot plant.

8. The recombinant expression construct of claim 2, wherein the terminator molecule is functionally linked to one or more promoter and one or more nucleic acid molecule, wherein the one or more promoter and/or the one or more nucleic acid molecule is heterologous to said one or more terminator.

9. A recombinant expression vector comprising one or more recombinant expression construct of claim 8.

10. A transgenic plant or part thereof comprising one or more heterologous terminator molecule comprising the heterologous terminator molecule as defined in claim 2 a) to e).

11. A transgenic cell or transgenic callus comprising the recombinant expression vector of claim 9.

12. The transgenic cell or transgenic callus of claim 11, selected or derived from bacteria, fungi, yeasts, or plants.

13. A transgenic cell culture, transgenic seed, transgenic plant or part or propagation material or progeny derived therefrom comprising i) the heterologous terminator as defined in claim 2 a) to e); or ii) the recombinant expression construct of claim 2.

14. A method for the production of an agricultural product comprising
a) introducing a terminator molecule as defined in claim 2 a) to e) into a plant cell, plant or part thereof;
b) growing the plant cell, plant or part thereof; and
c) harvesting and processing the plant cell, plant or part thereof.

15. A method for the production of an agricultural product comprising
a) introducing the recombinant construct of claim 8 into a plant cell, plant or part thereof;
b) growing the plant cell, plant or part thereof; and
c) harvesting and processing the plant cell, plant or part thereof.

16. A method for the production of an agricultural product comprising
a) introducing the recombinant vector of claim 9 into a plant cell, plant or part thereof;
b) growing the plant cell, plant or part thereof; and
c) harvesting and processing the plant cell, plant or part thereof.

17. A method for the production of an agricultural product comprising
a) providing the transgenic plant of claim 13;
b) growing the plant cell, plant or part thereof; and
c) harvesting and processing the plant cell, plant or part thereof.


SEQUENCE LISTING (3 sequences)

SEQ ID NO: 1 - DNA, 860 bp, Zea mays
tagcttgaaaattgtattgtggggtcgtgtaagtggttaataaggggggcataggtacgtacttgtctgtcgccccagcgtgtgttggagacggtgaatcaggtgatgtgtacatgctgcttcactgtagtgtatgtgtatgtgtatgtagtaaataattccaggtatataaggcacgacttgtgtagtatcatatctggttctgacttctgattatctaaagggaaggattcgaatgtctggatttggatgtctgaagattcgaattaacatcagaatgtttcctaatgaagtgctaatatagatactatggatgttaaatggacgtatccgacgaatttggtggtttaagatttcgacaaaattcataaaaccatttaaaacttattgttaatactaattacaaataattttaaattttaataatatactagaaaactgatgtacaccatttaaaatattgaaaaatatttatattataaataaataattatgttaatcttacattagtaggagtatcataggaatttttagaatctaaaatacaaatatatttaatatacaaaagaatatatttagagtctaaaatataaatatacctggtataaataatttatggaaaaatatattcataatttgtttgtaatatgatttttaccaactataaatacatcgataaaacaaattaatacttattatgatgttatgtttcaaatcaaataagtgtccaaatatgactcgaattcaaagtattcgttcatatggattcgttttcatatgtattcgatccgaattcatccctatctatttcgtactcgcattatcgcattaccagttaagggatgttccgattttttggt

SEQ ID NO: 2 - DNA, 471 bp, Zea mays
taggtagctaccgtacatgttttctgtaccttggaagtagtgcgtactgcagctgatgtattttcatgcatgccaactagactagctagtagttactgtacgtatgtagttgatagtgtaatcaggccgggtagcagctagctagaccaacttaattagctaatgtagtttgatgtacctgtctgtcagtctgtgtatgtttcgttgttttaatcgaccaacttgttccgtgtcctttttgttgtctagttctgtctactgttctgtttgtctgattggatccatcagcccatccatgtaccgcctggctagcttatcaggtattgcaatccaattcaggaactcacctctatctccttttctcagctttttctgtaaacaggatccgcacttttgctacaaaacaagttagattcactacagtaaactaacaaacatccgactgccgccttatctccgacgggtgtgtac

SEQ ID NO: 3 - DNA, 253 bp, Agrobacterium tumefaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc